(19) United States
(12) Patent Application Publication
(10) Pub. No.: US 2009/0012118 A1
(43) Pub. Date: Jan. 8, 2009

(54) Kynurenic Acid Amide Derivatives as Nr2b Receptor Antagoni

(76) Inventors: István Borza, Budapest (HU); Csilla Horvath, Budapest (HU); Sandor Farkas, Budapest (HU); Jozsef Nagy, Budapest (HU); Sandor Kolok, Budapest (HU)

Correspondence Address: K.F. ROSS P.C., 5683 RIVERDALE AVENUE, SUITE 203 BOX 900, BRONX, NY, 10471-0900, US

(21) Appl. No.: 11/658,690

(22) PCT Filed: Jul. 21, 2005
(86) PCT No.: PCT/HU05/00080

§ 371 Date: Jan. 25, 2007

(30) Foreign Application Priority Data
Jul. 29, 2004 (HU) P041525

(51) Int. Cl. A61K 31/4709; C07D 401/10; A61P 25/00

(52) U.S. Cl. 514/312; 546/156

(57) ABSTRACT

The new kynurenic acid amide derivatives of formula (I): and optical antipodes, racemates and the salts thereof are highly effective and selective antagonists of NMDA receptor, and moreover most of the compounds are selective antagonist of NR2B subtype of NMDA receptor.

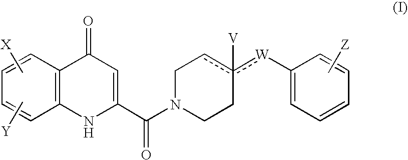

The invention relates to new kynurenic acid amide derivatives which are antagonists of NMDA receptor or are intermediates for preparing thereof.

### BACKGROUND OF THE INVENTION

N-methyl-D-aspartate (NMDA) receptors are ligand-gated cation-channels embedded in the cell membranes of neurones. Overactivation of NMDA receptors by glutamate, their natural ligand, can lead to calcium overload of cells. This triggers a cascade of intracellular events that alters the cell function and ultimately may lead to death of neurons [TINS, 10, 299-302 (1987)]. Antagonists of the NMDA receptors may be used for treating many disorders that are accompanied with excess release of glutamate, the main excitatory neurotransmitter in the central nervous system.

The NMDA receptors are heteromeric assemblies built up from at least 7 known subunit genes. The NR1 subunit is a necessary component of functional NMDA receptor channels. There are four genes encoding NR2 subunits (NR2A-D). Both spatial distributions in the CNS and the pharmacological sensitivity of NMDA receptors built up from various NR2 subunits are different. Recently, NR3A and NR3B have been reported. Particularly interesting of these is the NR2B subunit due to its restricted distribution (highest densities in the forebrain and substantia gelatinosa of the spinal cord). Compounds selective for this subtype are available and have been proved to be effective in animal models of stroke [Stroke, 28, 2244-2251 (1997)], traumatic brain injury [Brain Res., 792, 291-298 (1998)], Parkinson's disease [Exp. Neurol., 163, 239-.243 (2000)], neuropathic and inflammatory pain [Neuropharmacology, 38, 611-623 (1999)]. Moreover, NR2B subtype selective antagonists of NMDA receptors are expected to possess little or no untoward side effects that are typically caused by the non-selective antagonists of NMDA receptors, namely psychotomimetic effects such as dizziness, headache, hallucinations, dysphoria and disturbances of cognitive and motor function.

NR2B subtype selective NMDA antagonism can be achieved with compounds that specifically bind to, and act on, an allosteric modulatory site of the NR2B subunit containing receptors. This binding site can be characterised by displacement (binding) studies with specific radioligands, such as [¹²⁵I]-ifenprodil [J. Neurochem., 61, 120-126 (1993)] or [³H]-Ro 25,6981 [J. Neurochem., 70, 2147-2155 (1998)]. Since ifenprodil was the first, though not sufficiently specific, known ligand of this receptor, it has also been termed ifenprodil binding site.

Close structure analogs of the carboxylic acid amide derivatives of formula (I) are. unknown from the literature.

### SUMMARY OF THE INVENTION

Surprisingly it was found that the new kynurenic acid amide derivatives of formula (I) of the present invention are functional antagonists of NR2B subunit containing NMDA receptors, while they are ineffective on NR2A subunit containing NMDA receptors. Therefore, they are believed to be NR2B subtype specific NMDA antagonists. Some compounds proved to be effective in vivo in mouse pain model after oral administration.

### DETAILED DESCRIPTION OF THE INVENTION

The present invention relates therefore first to new kynurenic acid amide derivatives of formula (I)

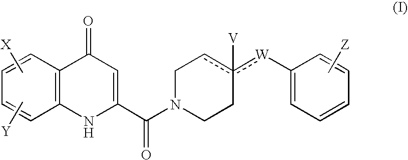

wherein the meaning of

X and Y independently are hydrogen atom, hydroxy, amino, C₁-C₄ alkylsulfonamido optionally substituted with a halogen atom or halogen atoms, C₁-C₄ alkanoylamido optionally substituted with a halogen atom or halogen atoms, C₁-C₄ alkoxy, C₁-C₄ alkoxycarbonyl group, or

the neighboring X and Y groups can form in given case together with one or more identical or different additional hetero atom and —CH═ and/or —CH₂— groups an optionally substituted 47 membered homo- or heterocyclic ring, preferably morpholine, pyrrole, pyrrolidine, oxo- or thioxo-pyrrolidine, pyrazole, pyrazolidine, imidazole, imidazolidine, oxo- or thioxo-imidazole or imidazolidine, 1,4-oxazine, oxazole, oxazolidine, oxo- or thioxo-oxazolidine, or 3-oxo-1,4-oxazine ring,

W is oxygen atom, as well as C₁-C₄ alkylene, C₂-C₄ alkenylene, aminocarbonyl, —NH—, —N(alkyl)—, —CH₂O—, —CH₂S—, —CH(OH)—, —OCH₂— group, —wherein the meaning of alkyl is a C₁-C₄ alkyl group —,

when the dotted bonds ( [symbol] ) represent a simple C—C bond then the meaning of V is hydroxy group or hydrogen atom or

when W is C₁-C₄ alkylene or C₃-C₄ alkenylene group, then one of the dotted bonds ( [symbol] ) can represent a further double C—C bond and in this case V means an electron pair, which participate in the double bond,

Z is hydrogen or halogen atom, C₁-C₄ alkyl, C₁-C₄ alkoxy, trifluoromethyl, hydroxy or carboxyl group

and optical antipodes, racemates and the salts thereof.

Further objects of the invention are the processes for producing kynurenic acid amide derivatives of formula (I), and the pharmaceutical manufacture of medicaments containing these compounds, as well as the process of treatments with these compounds, which means administering to a mammal to be treated—including human—effective amount/amounts of kynurenic acid amide derivatives of formula (I) of the present invention as such or as medicament.

The new kynurenic acid amide derivatives of formula (I) of the present invention are highly effective and selective antagonists of NMDA receptor, and moreover most of the compounds are selective antagonist of NR2B subtype of NMDA receptor.

According to the invention the kynurenic acid amide derivatives of formula (I) are synthesized by reacting a carboxylic acid of formula (II)

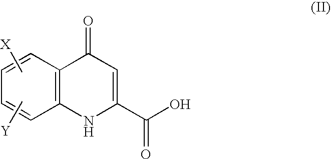

wherein the meaning of X and Y are as described before for the formula (I)—or an active derivative thereof with an amine of formula (III)

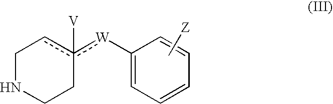

wherein the meaning of Z, V, W and the dotted bonds ( [symbol] ) are as given before for the formula (I),

then the so obtained kynurenic acid amide derivatives of formula (I)—wherein the meaning of X, Y, W, Z and the dotted bonds ( [symbol] ) are as defined for the formula (I)—in given case are transformed into another compounds of formula (I) by introducing new substituents and/or modifying or removing the existing ones, and/or by forming salt and/or-by liberating the compound from salts, and/or by resolving the obtained racemates using optically active acids or bases by known methods.

The reaction of the carboxylic acid of formula (II) and the amine of formula (III), i.e. the amide bond formation is preferably carried out by preparing an active derivative from the carboxylic acid of formula (II) and this is reacted with an amine of formula (III) preferably in the presence of a base.

The transformation of a carboxylic acid into an active derivative is carried out in situ during the amide bond formation in a proper solvent (for example dimethylformamide, acetonitrile, chlorinated hydrocarbons or hydrocarbons). The active derivatives can be acid chlorides (for example prepared from carboxylic acid with thionyl chloride), mixed anhydrides (for example prepared from carboxylic acid with isobutyl chloroformate in the presence of a base, e.g. triethylamine), active esters (for example prepared from carboxylic acid with hydroxybenztriazol and dicyclohexyl-carbodiimide or O-benzotriazol-1-yl-N,N,N′,N′-tetramethyluronium hexafluorophosphate (HBTU) in the presence of a base e.g. triethylamine). The active derivatives are prepared between room temperature and 0° C. A proper amine of formula (III) is added as base or as a salt formed with inorganic acid to the so obtained solution or suspension so that base, for example triethylamine, needed for the liberation of the amine, is added to the reaction mixture separately. The condensation reactions are followed by thin layer chromatography. The necessary reaction time is 6-20 h. The work-up of the reaction mixture can be carried out by different methods.

When the reaction mixture is a suspension, the precipitate is filtered off and recrystallized from a proper solvent to give the pure product. If the crystallization does not lead to the pure product, then column chromatography can be used for the purification of it. The column chromatography is carried out on Kieselgel 60 as adsorbent using different solvent systems, e.g. toluene/methanol, chloroform/methanol or toluene/acetone, as eluents. If the reaction mixture is a solution at the end of the acylation, it is concentrated, and the residue is crystallized or purified by column chromatography as described above. The structures of the products are determined by IR, NMR and mass spectrometry.

The obtained kynurenic acid amide derivative of formula (I)—independently from the method of preparation—in given case can be transformed into an other kynurenic acid amide derivative of formula (I) by introducing further substituents and/or modifying and/or removing the existing ones, and/or formation of salts with acids and/or liberating the carboxylic acid amide derivative of formula (I) from the obtained acid addition salts by treatment with a base and/or the free kynurenic acid amide derivative of formula (I) can be transformed into a salt by treatment with a base.

For example cleaving the methyl and benzyl groups from methoxy and benzyloxy groups, which stands for X, Y and Z, leads to phenol derivatives. The removal of the benzyl group can be carried out for example with catalytic hydrogenation or with hydrogen bromide in acetic acid solution, the cleavage of methyl group can be carried out with boron tribromide in dichloromethane solution. The kynurenic acid amide derivatives of formula (I) containing free phenolic hydroxy group can be transformed into acyloxy group with different acylating agents. The reactions are carried out at room temperature in chlorinated hydrocarbons using acid chloride or acid anhydride as acylating agent in the presence of a base (for example triethylamine or sodium carbonate). The kynurenic acid amide derivatives of formula (I) containing an amino group can be transformed into acylamido or sulfamido derivatives with different acylating or sulfonylating agents described for the acylation of phenolic hydroxy groups. Free hydroxy groups can be esterified by acid anhydrides or acid halogenides in the presence of abase.

The carboxylic acids of formula (II) and the secondary amines of formula (III) are either commercially available or can be synthesized by different known methods. The syntheses of some commercially not available carboxylic acids of formula (II) and the secondary amines of formula (III) are described in the Examples.

### Experimental Protocols

### Expression of Recombinant NMDA Receptors

To prove NR2B selectivity of our compounds, we tested them on cell lines stably expressing recombinant NMDA receptors with subunit compositions of NR1/NR2A or NR1/NR2B. cDNAs of human NR1-3 and NR2A or rat NR1a and NR2B subunits subcloned into inducible mammalian expression vectors were introduced into HEK 293 cells lacking NMDA receptors using a cationic lipid-mediated transfection method [Biotechniques, 1997 May :22(5),: 982-7; Neurochemistry International, 43, 19-29. (2003)]. Resistance to neomycin and hygromycin was used to screen for clones possessing both vectors and monoclonal cell lines were established from the clones producing the highest response to NMDA exposure. Compounds were tested for their inhibitory action on NMDA evoked cytosolic calcium elevations in fluorescent calcium measurements. Studies were performed 48-72 h after addition of the inducing agent. Ketamine (500 μM) was also present during the induction in order to prevent cytotoxicity.

### Assessing the Functional NMDA Antagonist Potency of Compounds on HEK293 Cells Expressing Recombinant NMDA Receptors Based on Measuring the Intracellular Calcium Concentration Using a Fluorimeter Plate Reader

Since NMDA receptors are known to be permeable to calcium ions upon excitation, the extent of NMDA receptor activation, and its inhibition by functional antagonists can be characterized by measuring the rise in the intracellular calcium concentration following agonist (NMDA) application onto the cells. Since there is very high sequence homology between rat and human NMDA receptors (99, 95, 97% for NR1, NR2A, and NR2B subunits, respectively), it is believed that there is little, if any, difference in their pharmacological sensitivity. Hence, results obtained with (cloned or native) rat NMDA receptors may be well extrapolated to the human ones.

The intracellular calcium measurements are carried out on HEK293 cells expressing NR1a and NR2B or NR2A NMDA receptor subunits. The cells are plated onto standard 96-well microplates and the cultures are maintained in an atmosphere of 95% air −5% CO₂ at 37° C. until testing.

The cells are loaded with a fluorescent Ca²⁺-sensitive dye, Fluo-4/AM (2-2.5 μM) prior to testing. Loading is stopped by washing twice with the solution used also during the measurement (140 mM NaCl, 5 mM KCl, 2 mM CaCl₂, 5 mM HEPES [4-(2-hydroxyethyl)-1-piperazineethane-sulfonic acid], 5 mM HEPES-Na, 20 mM glucose, 10 μM glycine, pH=7.4). Then the test compound dissolved in the above solution (90 μl/well) is added. Intracellular calcium measurements are carried out with a plate reader fluorimeter. A rise in Fluo-4-fluorescence that reflects the intracellular calcium concentration is induced by application of 200 μM NMDA. Inhibitory potency of the test compound is assessed by measuring the reduction in the calcium elevation in the presence of different concentrations of the compound.

Inhibitory potency of a compound at a single concentration point is expressed as percent inhibition of the control NMDA response. For NR1a/NR2B expressing cells concentration-inhibition curves are produced. Sigmoidal concentration-inhibition curves are fitted over the data and IC₅₀ values are defined as the concentration that produces half of the maximal inhibition that could be achieved with the compound. Mean IC₅₀ values are derived from at least three independent experiments. For NR1-3/NR2A expressing cells antagonism of NMDA induced rise in intracellular calcium concentration by compounds of the present invention and reference compounds was tested at 10 and 15 microM concentration, respectively.

### The Biological Activity of the Compounds

IC₅₀ values determined in NR1a/NR2B transfected cells and percentage inhibition at 15 μM concentration in NR1a/NR2A transfected cells are listed in Table 1 for selected examples of compounds of this invention. For comparison, data for the most potent known reference compounds were also determined and are given in Table 2.

The compounds of this invention exhibit IC₅₀ values of less than 15 μM in the functional NMDA antagonism test in NR1-3/NR2A transfected cells, and are inactive at this concentration on NR1/NR1A transfected cells. Thus the compounds and pharmaceutical compositions of this invention are NR2B subtype specific NMDA antagonists. Some of the compounds have superior potency compared to the known reference compounds (see Table 1).

TABLE 1
NMDA antagonist activity of compounds measured by
fluorimetric method on cells expressing NR1a/NR2B
or NR1-3/NR2A subunits

| | | |
|---|---|---|
| Compound of | NR1a/NR2B | NR1-3/NR2A |

| Example | IC₅₀ [nM] | n | % inhibition at 15 μM | n |
|---|---|---|---|---|
| 2 | 3.7 | 3 | 7.0 | 1 |
| 1 | 4.0 | 2 | −0.9 | 1 |
| 3 | 3.8 | 2 | −2.4 | 1 |
| 5 | 21.5 | 2 | 5.7 | 1 |
| 6 | 8.9 | 2 | 14.0 | 1 |
| 4 | 1.9 | 2 | −5.1 | 1 |
| 7 | 5.1 | 2 | 5.1 | 1 |
| 8 | 3.3 | 2 | −3.5 | 1 |
| 9 | 5.6 | 2 | −2.1 | 1 |
| 10 | 28.0 | 2 | 0.2 | 1 |

TABLE 2
NMDA antagonist activity of reference compounds
measured by fluorimetric method on
cells expressing NR1a/NR2B or NR1-3/NR2A subunits

NR1-3/NR2A

| | | |
|---|---|---|
| Code of | NR1a/NR2B | % inhibition |

| reference compounds | IC₅₀ [nM] | n | at 10 μM | n |
|---|---|---|---|---|
| CI-1041 | 8.4 | 4 | 21.0 | 1 |
| Co-101244 | 4.8 | 3 | −8.7 | 1 |
| EMD 95885 | 48 | 1 | 0.1 | 1 |
| CP 101,606 | 30 | 3 | 2.5 | 1 |
| Ro 25.6981 | 57 | 4 | 1.0 | 1 |
| ifenprodil | 459 | 5 | −2.7 | 1 |
| MK-801 | 43 | 3 | IC₅₀ = 386 | 2 |
| | | | nM | |

The reference compounds are as follows:
CI-1041: 6-{2-[4-(4-fluoro-benzyl)-piperidin-1-yl]-ethanesulfinyl}-3H-benzooxazol-2-one
Co 101244: 1-[2-(4-hydroxyphenoxy)ethyl]-4-hydroxy-4-(4-methylbenzyl)piperidine
EMD 95885: 6-[3-(4-fluorobenzyl)piperidine-1-yl]propionyl]-2,3-dihydro-benzoxazol-2-on
CP-101,606: (1S,2S)-1-(4-hydroxyphenyl)-2-(4-hydroxy-4-phenylpiperidine-1-yl)-1-propanol
Co-111103: 1-[2-(4-hydroxyphenoxy)ethyl]-4-(4-fluorobenzyl)piperidine
Ro 256981: R—(R*,S*)-1-(4-hydroxyphenyl)-2-methyl-3-[4-(phenylmethyl)piperidin-1-yl]-1-propanol.
Ifenprodil: erythro-2-(4-benzylpiperidino)-1-(4-hydroxyphenyl)-1-propanol
MK-801: (+)-5-Methyl-10,11-dihydro-5H-dibenzo[a,d]cyclohepten-5,10-imine

### Mouse Formalin Test for Measurement of In Vivo Efficacy

Injection of diluted formalin into the hind paw of rats or mice is known to elicit a biphasic pain-related behavior measured as time spent by licking/biting of the injured paw. The second phase is generally defined as pain related events detected in the 15-60 min. time interval after formalin injection. It is known that NMDA receptors are involved in the second phase of response to formalin injection and this behavioral response is sensitive to blockade of NMDA receptors [Dickenson, A. and Besson J.-M. (Editors): Chapter 1, pp. 6-7: Animal models of Analgesia; and Chapter 8, pp. 180-183: Mechanism of Central Hypersensitivity: Excitatory Amino Acid Mechanisms and Their Control—In Pharmacology of Pain. Springer-Verlag (Berlin) 1997.] Therefore, we used the second phase of formalin test to characterize the efficacy of compounds in vivo. Inhibition of the second phase of response is considered to indicate an analgesic effect against chemically-induced persistent pain [Hunskaar, S., et al.: Formalin Test in Mice, a Useful Technique for Evaluating Mild Analgesics, Journal of Neuroscience Methods, 14 (1985) 69-76.]

Male albino NMRI mice (20-25 g) were used. Prior to the experiment any solid food was withdrawn for approx. 16 hours but the animals had free access to 20% glucose solution. The animals were allowed 1 hour acclimatization period in a glass cylinder (cc. 15 cm in diameter), then moved to an identical cylinder with a mirror placed behind to facilitate observation. The test substances were suspended in 5% tween-80 (10 ml per kg body weight). and administered orally by gavage 15 min before the formalin injection (20 μl of 1% formalin in 0.9% saline injected subcutaneously into the dorsal surface of the right hind paw). The time spent by licking and biting of the injected paw was measured from 20 to 25 min. after the formalin injection. For the determination of ED₅₀ value, various doses (at least five) of the test substances were given to groups of 5 mice and the results expressed as % inhibition time spent by licking relative to a vehicle control group observed on the same day. ED₅₀ values (i.e. the dose yielding 50% inhibition) were calculated by Boltzman's sigmoidal curve fitting.

TABLE 3
ED₅₀ values of selected compounds in mouse formalin test

| | ED₅₀ (mg/kg p.o.) |
|---|---|
| Compound of Example | |
| 2 | >20 |
| 1 | 9.5 |
| 3 | 8.9* |
| 4 | >20 |
| 6 | 0.46 |
| 7 | >20 |
| 8 | 8.2 |
| 9 | >20 |
| Code of | — |
| reference compounds | |
| CI-1041 | 2.4 mg/kg |
| Co-101,244 | p.o. inactive, 5.9 mg/kg i.p. |
| EMD 95885 | 3.7 mg/kg |
| CP-101,606 | >20 mg/kg (s.c. and p.o.) |
| Co-111103 | >20 mg/kg |
| Ro-25,6981 | p.o. inactive, 5.1 mg/kg i.p. |

*partial inhibition with 60% maximal effect

Disorders which may be beneficially treated with NMDA antagonists acting at NR2B site, as reviewed recently by Loftis [Pharmacology & Therapeutics 2003, 97: 55-85] include schizophrenia, Parkinson's disease, Huntington's disease, excitotoxicity evoked by hypoxia and ischemia, seizure disorders, drug abuse, and pain, especially neuropathic, inflammatory and visceral pain of any origin [Eur. J. Pharmacol. 2001, 429: 71-78].

Due to their reduced side effect liability compared to non-selective NMDA antagonists, NR2B selective antagonists may have utility in diseases where NMDA antagonist may be effective, such as amyotrophic lateral sclerosis [Neurol. Res., 21, 309-12 (1999)], withdrawal syndromes of e.g. alcohol, opioids or cocaine [Drug and Alcohol Depend., 59, 1-15 (2000)], muscular spasm [Neurosci. Lett., 73, 143-148 (1987)], dementia of various origins. [Expert Opin. Investig. Drugs, 9, 1397-406 (2000)], anxiety, depression, migraine, hypoglycemia, degenerative disorders of the retina (e.g. CMV retinitis), glaucoma, asthma, tinnitus, hearing loss [Drug News Perspect 11, 523-569 (1998) and WO 00/00197 international patent application].

Accordingly, effective amounts of the compounds of the invention may be beneficially used for the treatment of traumatic injury of brain or spinal cord, tolerance and/or dependence to opioid treatment of pain, development of tolerance, decrease of abuse potential and withdrawal syndromes of drugs of abuse e.g. alcohol, opioids or cocaine, ischemic CNS disorders, chronic neurodegenerative disorders, such as e.g. Alzheimer's disease, Parkinson's disease, Huntington's disease, pain and chronic pain states, such as e.g, neuropathic pain.

The compounds of the invention as well as their pharmaceutically acceptable salts can be used as such or suitably in the form of pharmaceutical compositions. These compositions (drugs) can be in solid, liquid or semiliquid form and pharmaceutical adjuvant and auxiliary materials can be added, which are commonly used in practice, such as carriers, excipients, diluents, stabilizers, wetting or emulsifying agents, pH- and osmotic pressure-influencing, flavoring or aromatizing, as well as formulation-promoting or formulation-providing additives.

The dosage required to exert the therapeutical effect can vary within wide limits and will be fitted to the individual requirements in each of the particular cases, depending on the stage of the disease, the condition and the bodyweight of the patient to be treated, as well as the sensitivity of the patient against the active ingredient, route of administration and number of daily treatments. The actual dose of the active ingredient to be used can safely be determined by the attending physician skilled in the art in the knowledge of the patient to be treated.

The pharmaceutical compositions containing the active ingredient according to the present invention usually contain 0.01 to 100 mg of active ingredient in a single dosage unit. It is, of course possible that the amount of the active ingredient in some compositions exceeds the upper or lower limits defined above.

The solid forms of the pharmaceutical compositions can be for example tablets, dragées, capsules, pills br lyophilized powder ampoules useful for the preparation of injections. Liquid compositions are the injectable and infusable compositions, fluid medicines, packing fluids and drops. Semiliquid compositions can be ointments, balsams, creams, shaking mixtures and suppositories.

For the sake of a simple administration it is suitable if the pharmaceutical compositions comprise dosage units containing the amount of the active ingredient to be administered once, or a few multiples or a half, third or fourth part thereof. Such dosage units are e.g. tablets, which can be powdered with grooves promoting the halving or quartering of the tablet in order to exactly administer the required amount of the active ingredient.

Tablets can be coated with an acid-soluble layer in order to assure the release of the active ingredient content after leaving the stomach. Such tablets are enteric-coated. A similar effect can be achieved also by encapsulating the active ingredient.

The pharmaceutical compositions for oral administration can contain e.g. lactose or starch as excipients, sodium carboxymethylcellulose, methylcellulose, polyvinyl pyrrolidine or starch paste as binders or granulating agents. Potato starch or microcrystalline cellulose is added as disintegration agents, but ultraamylopectin or formaldehyde casein can also be used. Talcum, colloidic silicic acid, stearin, calcium or magnesium stearate can be used as antiadhesive and lubricants.

The tablet can be manufactured for example by wet granulation, followed by pressing. The mixed active ingredients and excipients, as well as in given case part of the disintegrants are granulated with an aqueous, alcoholic or aqueous alcoholic solution of the binders in an appropriate equipment, then the granulate is dried. The other disintegrants, lubricants and antiadhesive agents are added to the dried granulate, and the mixture is pressed to a tablet. In given case the tablets are made with halving groove to ease the administration.

The tablets can be made directly from the mixture of the active ingredient and the proper auxiliaries by pressing. In given case, the tablets can be coated by using additives commonly used in the pharmaceutical practice, for example stabilizers, flavoring, coloring agents, such as sugar, cellulose derivatives (methyl- or ethylcellulose, sodium carboxymethylcellulose, etc), polyvinyl pyrrolidone, calcium phosphate, calcium carbonate, food coloring agents, food laces, aroma agents, iron oxide pigments, etc. In the case of capsules the mixture of the active ingredient and the auxiliaries is filled into capsules.

Liquid oral compositions, for example suspensions, syrups, elixirs can be made by using water, glycols, oils, alcohols, coloring and flavoring agents.

For rectal administration the composition is formulated in suppositories or clysters. The suppository can contain beside the active ingredient a carrier, so called adeps pro suppository. Carriers can be vegetable oils, such as hydrogenated vegetable oils, triglycerides of C₁₂-C₁₈ fatty acids (preferably the carriers under the trade name Witepsol). The active ingredient is homogeneously mixed with the melted adeps pro suppository and the suppositories are moulded.

For parenteral administration the composition is formulated as injection solution. For manufacturing the injection solution the active ingredients are dissolved in distilled water and/or in different organic solvents, such as glycol ethers, in given case in the presence of solubilizers, for example polioxyethylensorbitane-monolaurate, -monooleate, or monostearate (Tween 20, Tween 60, Tween 80). The injection solution can also contain different auxiliaries, such as conserving agents, for example ethylendiamine tetraacetate, as well as pH adjusting agents and buffers and in given case local anesthetic, e.g. lidocain. The injection solution containing the active ingredient of the invention is filtered before it is filled into ampoules, and it is sterilized after filling.

If the active ingredient is hygroscopic, then it can be stabilized by liophylization.

The following examples illustrate the invention without the intention of limitation anyway.

### EXAMPLE 1

### 2-[4-(4-Fluoro-benzyl)-piperidine-1-carbonyl]-6-hydroxy-1H-quinolin-4-one

A mixture of 0.5 g (2.4 mmol) of 6-hydroxy4-oxo-1,4-dihydro-quinoline-2-carboxylic acid [J. Med. Chem., 17, 685-690. (1974)], 0.75 ml (5.4 mmol) of triethylamine, 0.6 g (2.6 mmol) of 4-(4-fluoro-benzyl)-piperidine hydrochloride [J. Med. Chem., 35, 4903. (1992)], 1.0 g (2.6 mmol) of HBTU [O-benzotriazol-1-yl-N,N,N′,N′-tetramethyluronium hexafluorophosphate (Advanced Chem. Tech.)] and 15 ml of dimethylformamide is stirred at room temperature for 24 h. The reaction mixture is concentrated and the residue is purified by column chromatography using Kieselgel 60 as adsorbent (Merck) and toluene:methanol=4:1 as eluent to yield 0.18 g (19%) of the title compound. Mp.: 190° C. (diethylether).

### EXAMPLE 2

### 2-(4-Benzyl-piperidine-1-carbonyl)-6-hydroxy-1H-quinolin-4-one

The title compound is prepared from 6-hydroxy-4-oxo-1,4-dihydro-quinoline-2-carboxylic acid and 4-benzyl-piperidine according to the method described in Example 1. Mp.: 127° C. (diethylether).

### EXAMPLE 3

### 6-Hydroxy-2-[4-(4-methyl-benzyl)-piperidine-1-carbonyl]-1H-quinolin-4-one

The title compound is prepared from 6-hydroxy-4-oxo-1,4-dihydro-quinoline-2-carboxylic acid and 4-(4-methylbenzyl)-piperidine [J Org. Chem., 64, 3763. (1999)] according to the method described in Example 1. Mp.: 152° C. (diethylether).

### EXAMPLE 4

### 2-[4-(4-Chloro-benzyl)-piperidine-1-carbonyl]-6-hydroxy-1H-quinolin-4-one

The title compound is prepared from 6-hydroxy-4-oxo-1,4-dihydro-quinoline-2-carboxylic acid and 4-(4-chloro-benzyl)-piperidine (C. A. 77, 34266 w) according to the method described in Example 1. Mp.: 194° C. (diethylether).

### EXAMPLE 5

### 2-(4-Benzyloxy-piperidine-1-carbonyl)-6-hydroxy-1H-quinolin-4-one

The title compound is prepared from 6-hydroxy4-oxo-1,4-dihydro-quinoline-2-carboxylic acid and 4-benzyloxy-piperidine [Tetrahedron Lett., 36, 3465. (1995)] according to the method described in Example 1. Mp.: 103° C. (diethylether).

### EXAMPLE 6

### 6-Hydroxy-2-(4-Phenoxymethyl-piperidine-1-carbonyl)-1H-quinolin-4-one

The title compound is prepared from 6-hydroxy-4-oxo-1,4-dihydro-quinoline-2-carboxylic acid and 4-phenoxy-methyl-piperidine [DE 254 999 (1977)] according to the method described in-Example 1. Mp.: 130° C. (diethylether).

### EXAMPLE 7

### 2-[4-(4-Chloro-phenoxy-piperidene-1-carbonyl]-6hydroxy-1H-quinolin-4-one

a) 4-(4-Chloro-phenoxy)-piperidine-1-carboxylic Acid Tert-Butyl Ester

Under argon, to a stirred solution of 10.0 g (49.7 mmol) of 4-hydroxy-piperidin-1-carboxylic acid tert-butyl ester [Bioorg. Med. Chem. Lett. 10, 2815. (2000)] in 80 ml of dimethylformamide 3.0 g (60%, 75 mmol) of sodium hydride is added. The reaction mixture is stirred at 40° C. for 1 h, then 5.3 ml (49.7 mmol) of 1-chloroofluoro-benzene (Aldrich) in 20 ml of dimethylformamide is added drop wise at 20° C. The reaction mixture is stirred at 80° C. for 4 h, cooled to 20° C. 1 ml of ethanol is added drop wise, poured into 100 ml of water and extracted with ethyl acetate. The organic layer is dried over sodium sulfate and concentrated. The residue is purified by column chromatography using Kieselgel 60 (Merck) as adsorbent and ethyl acetate as eluent to yield 11.07 g (75.5%) of the title compound. Mp.: oil.

b) 4-(4-Chloro-phenoxy)-piperidine Hydrochloride

To a solution of 150 ml of 2.5 M hydrochloric acid in ethyl acetate 11.07 g (37.5 mmol) of 4-(4-chloro-phenoxy)-piperidin-1-carboxylic acid tert-butyl ester is added. The reaction mixture is stirred at 20° C. for 3 h, then concentrated to 50 ml. The precipitated crystals are filtered off, washed with ethyl acetate to yield 7.0 g (75.2%) of the title compound. Mp.: 194-196° C.

c) 2-[4-(4-Chloro-phenoxy)-piperidine-1-carbonyl]-6-hydroxy-1H-quinolin4-one (45

The title compound is prepared from 6-hydroxy-4-oxo-1,4-dihydro-quinoline-2-carboxylic acid and 4-(4-chloro-phenoxy)-piperidine according to the method described in Example 1. Mp.: 91° C. (diethylether).

### EXAMPLE 8

### 6-Hydroxy-2-(4-p-tolyloxy-piperidine-1-carbonyl)-1H-quinolin4-one

The title compound is prepared from 6-hydroxy4-oxo-1,4-dihydro-quinoline-2-carboxylic acid and 4-p-tolyloxy-piperidine [J Med. Chem., -21, 309. (1978)] according to the method described in Example 1. Mp;: 258-260° C. (diethylether).

### EXAMPLE 9

### 6-(4-Benzyl-piperidine-1-carbonyl)-1,5-dihydro-oxazolo[4,5-g]quinoline-2,8-dione

a) 2-(2-Oxo-2,3-dihydro-benzooxazol-6-ylamino)-but-2-enedioic Acid Dimethyl Ester

A mixture of 1.0 g (6.66 mmol) of 6-amino-3H-benzooxazol-2-one [U.S. Pat. No. 2,806,853], 0.9 ml (7.3 mmol) of dimethyl acetylenedicarboxylate (Aldrich) and 15 ml of methanol is refluxed for 2 h. The reaction mixture is cooled to 20° C., the precipitated crystals are filtered off, washed with methanol to yield 1.7 g (87%) of the title compound. Mp.: 172° C.

b) 2,8-Dioxo-1,2,5,8-tetrahydro-oxazolo[4,5-g]quinoline-6-carboxylic Acid Methyl Ester

To a stirred solution of 10 ml of boiling Dowtherm (Fluka) 1.7 g (5.8 mmol) of 2-(2-oxo-2,3-dihydro-benzooxazol-6-ylamino)-but-2-enedioic acid dimethyl ester is added in small portions. After completion of the addition the reaction mixture is refluxed for 10 min., then cooled to room temperature, the precipitated product is filtered off and washed with hexane to yield 1.16 g (76%) of the title compound. Mp.: 297° C.

c) 2.8-Dioxo-1,2,5,8-tetrahydro-oxazolo[4,5-g]quinoline-6-carboxylic Acid

A mixture of 1.16 g (4;4 mmol) of 2-8-dioxo-1,2,5,8-tetrahydro-oxazolo[4,5-g]quinoline-6-carboxylic acid methyl ester, 40 ml of methanol, 10 ml of water and 1.25 g (31.2. mmol) of sodium hydroxide is stirred at 20° C. for 1 h. The methanol is distilled off under reduced pressure. The reaction mixture is acidified with 2M hydrochloric acid and the precipitated crystals are filtered off, washed with water to yield 0.9 g (82%) of the title compound. Mp.>300° C.

d) 6-(4-Benzyl-piperidine-1-carbonyl)-1,5-dihydro-oxazolo[4,5-g]quinoline-2,8-dione

The title compound is prepared from 2,8-dioxo-1,2,5,8-tetrahydro-oxazolo[4,5-g]quinoline-6-carboxylic acid and 4-benzyl-piperidine according to the method described in Example 1. Mp.: 215° C. (diethylether).

### EXAMPLE 10

### 6-Hydroxy-2-(4-phenoxy-piperidine-1-carbonyl)-1H-quinolin-4-one

The title compound is prepared from 6-hydroxy-4-oxo-1,4-dihydro-quinoline-2-carboxylic acid and 4-phenoxy-piperidine [J Med. Chem., 17, 1000-1003. (1974)] according to the method described in Example 1. Mp.: 270° C. (diethylether).

### EXAMPLE 11

### 2-(4-Benzyl-4-hydroxy-piperidine-1-carbonyl)-6-hydroxy-1H-quinolin-4-one

The title compound is prepared from 6-hydroxy-4-oxo-1,4-dihydro-quinoline-2-carboxylic acid and 4benzyl-piperidin-4-ol [J Med. Chem., 42, 2087-2104. (1999)] according to the method described in Example 1. Mp.: 178° C. (diethylether).

### EXAMPLE 12

### 2-(4-1Benzyl-4-hydroxy-piperidine-1-carbonyl)-7-hydroxy-1H-quinolin-4-one

a) 2-(4-Benzyl-piperidine-1-carbonyl)-7-benzyloxy-1H-quinolin-4-one

The title compound is prepared from 7-benzyloxy-4-oxo-1,4-dihydro-quinoline-2-carboxylic acid [( Med. Chem., 34, 1243-1252. (1991)] and 4-benzyl-piperidine according to the method described in Example 1. Mp.: 228° C. (izopropanol).

b.) 2-(4-Benzyl-4-hydroxy-piperidine-1-carbonyl)-7-hydroxy-1H-quinolin-4-one

A mixture of 0.5 g (1.1 mmol) of 2-(4-benzyl-piperidine-1-carbonyl)-7-benzyloxy-1H-quinolin-4-one, 20 ml of tetrahydrofuran, 20 ml of methanol, 0.2 g of 10% Pd/C catalyst is hydrogenated for 2 h. The catalyst is filtered off, washed with tetrahydrofuran and the filtrate is concentrated. The residue is purified by column chromatography using Kieselgel 60 as absorbent (Merck) and toluene: metanol=4:1 as eluent to yield 0.32 g (80.7%) of the title compound. Mp.: 174+ C. (diethylether).

### EXAMPLE 13

### Preparation of Pharmaceutical Compositions

a) Tablets:

0.01-50% of active ingredient of formula I, 15-50% of lactose, 15-50% of potato starch, 5-15% of polyvinyl pyrrolidone, 1-5% of talc, 0.01-3% of magnesium stearate, 1-3% -of colloid silicon dioxide and 2-7% of ultramylopectin are mixed, then are granulated by wet granulation and pressed to tablets.

b) Dragées, Filmcoated Tablets:

The tablets made according to the method described above are coated by a layer consisting of entero- or gastrosolvent film, or of sugar and talc. The dragées are polished by a mixture of beeswax and carnuba wax.

c) Capsules:

0.01-50% of active ingredient of formula I, 1-5% of sodium lauryl sulfate, 15-50% of starch, 15-50% of lactose, 1-3% of colloid silicon dioxide and 0.01-3% of magnesium stearate are thoroughly mixed, the mixture is passed through a sieve and filled in hard gelatin capsules.

d) Suspensions:

Ingredients: 0.01-15% of active ingredient of formula I, 0.1-2% of sodium hydroxide, 0.1-3% of citric acid, 0.05-0.2% of nipagin (sodium methyl 4-hydroxybenzoate), 0.005-0.02% of nipasol, 0.01-0.5% of carbopol (polyacrilic acid), 0.1-5% of 96% ethanol, 0.1-1% of flavoring agent, 20-70% of sorbitol (70% aqueous solution) and 30-50% of distilled water.

To solution of nipagin and citric acid in 20 ml of distilled water, carbopol is added in small portions under vigorous stirring, and the solution is left to stand for 10-12 h. Then the sodium hydroxide in 1 ml of distilled water, the aqueous solution of sorbitol and finally the ethanolic raspberry flavor are added with stirring. To this carrier the active ingredient is added in small portions and suspended with an immersing homogenizator. Finally the suspension is filled up to the desired final volume with distilled water and the suspension syrup is passed through a colloid milling equipment.

e) Suppositories:

For each suppository 0.01-15% of active ingredient of formula I and 1-20% of lactose are thoroughly mixed, then 50-95% of adeps pro suppository (for example Witepsol 4) is melted, cooled to 35° C. and the mixture of active ingredient and lactose is mixed in it with homogenizator. The obtained mixture is mould in cooled forms.

f) Lyophilized Powder Ampoule Compositions:

A 5% solution of mannitol or lactose is made with bidistilled water for injection use, and the solution is filtered so as to have sterile solution. A 0.01-5% solution of the active ingredient of formula I is also made with bidistilled water for injection use, and this solution is filtered so as to have sterile solution. These two solutions are mixed under aseptic conditions, filled in 1 ml portions into ampoules, the content of the ampoules is lyophilized, and the ampoules are sealed under nitrogen. The contents of the ampoules are dissolved in sterile water or 0.9% (physiological) sterile aqueous sodium chloride solution before administration

What we claim is:

1. New kynurenic acid amide derivatives of formula (I) 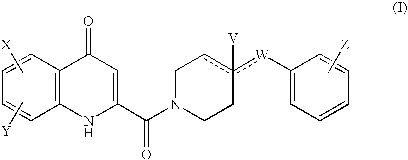
wherein the meaning of
X and Y independently are hydrogen atom, hydroxy, amino, C₁-C₄ alkylsulfonamido optionally substituted with a halogen atom or halogen atoms, C₁-C₄ alkanoylamido optionally substituted with a halogen atom or halogen atoms, C₁-C₄ alkoxy, C₁-C₄ alkoxycarbonyl group, or
the neighboring X and Y groups can form in given case together with one or more identical or different additional hetero atom and —CH═ and/or —CH₂— groups an optionally substituted 4-7 membered homo- or heterocyclic ring, preferably morpholine, pyrrole, pyrrolidine, oxo- or thioxo-pyrrolidine, pyrazole, pyrazolidine, imidazole, imidazolidine, oxo- or thioxo-imidazole or imidazolidine, 1,4-oxazine, oxazole, oxazolidine, oxo- or thioxo-oxazolidine, or 3-oxo-1,4-oxazine ring,
W is oxygen atom, as well as C₁-C₄ alkylene, C₂-C₄ alkenylene, aminocarbonyl, —NH—, —N(alkyl)—, —CH₂O—, —CH₂S—, —CH(OH)—, —OCH₂— group, —wherein the meaning of alkyl is a C₁-C₄ alkyl group—,
when the dotted bonds ( [symbol] ) represent a simple C-C bond then the meaning of V is hydroxy group or hydrogen atom or
when W is C₁-C₄ alkylene or C₃-C₄ alkenylene group, then one of the dotted bonds ( [symbol] ) can represent a further double C—C bond and in this case V means an electron pair, which participate in the double bond,
Z is hydrogen or halogen atom, C₁-C₄ alkyl, C₁-C₄ alkoxy, trifluoromethyl, hydroxy or carboxyl group
and optical antipodes, racemates and the salts thereof.

2. Compounds of formula (I) as defined in claim 1
wherein the meaning of
X is hydrogen atom and
Y is hydroxy or benzyloxy group, or
the neighboring X and Y groups form a —NH—CO—O— chain
W is oxygen atom, as well as C₁-C₄ alkylene, —CH₂O—, or —OCH₂— group,
V is hydroxy group or hydrogen atom,
Z is hydrogen or halogen atom, or C₁-C₄ alkyl group and
the dotted bonds ( [symbol] ) represent simple C—C bonds.

3. One compound of the following group of kynurenic acid amide derivatives belonging to the scope of claim 1
2-[4-(4-fluoro-benzyl)-piperidine-1-carbonyl]-6-hydroxy-1H-quinolin-4-one,
2-(4-benzyl-piperidine-1-carbonyl)-6-hydroxy-1H-quinolin-4-one,
6-hydroxy-2-[4-(4-methyl-benzyl)-piperidine-1-carbonyl]-1H-quinolin-4-one,
2-[4-(4-chloro-benzyl)-piperidine-1-carbonyl]-6-hydroxy-1H-quinolin-4-one,
2-(4-benzyloxy-piperidine-1-carbonyl)-6-hydroxy-1H-quinolin-4-one,
6-hydroxy-2-(4-phenoxymethyl-piperidine-1-carbonyl)-1H-quinolin4-one,
2-[4-(4-chloro-phenoxy)-piperidine-1-carbonyl]-6-hydroxy-1H-quinolin-4-one,
6-hydroxy-2-(4-p-tolyloxy-piperidine-1-carbonyl)-1H-quinolin-4-one,
6-(4-benzyl-piperidine-1-carbonyl)-1,5-dihydro-oxazolo[4,5-g]quinoline-2,8-dione,
6-hydroxy-2-(4-phenoxy-piperidine-1-carbonyl)-1H-quinolin-4-one,
2-(4-3Benzyl-4-hydroxy-piperidine-1-carbonyl)-6-hydroxy-1H-quinolin-4-one, and
2-(4-Benzyl-4-hydroxy-piperidine-1-carbonyl)-7-hydroxy-1H-quinolin4-one.

4. Pharmaceutical compositions containing an effective amount of the kynurenic acid amide derivatives of formula (1)—wherein the meaning of X, Y, W, V, Z and the dotted bonds ( [symbol] ) are as given in claim 1—or optical antipodes or racemates or the salts thereof as active ingredients and auxiliary materials, which are commonly used in practice, such as carriers, excipients, diluents, stabilizers, wetting or emulsifying agents, pH- and osmotic pressure-influencing, flavoring or aromatizing, as well as formulation-promoting or formulation-providing additives.

5. Process for preparing the kynurenic acid amide derivatives of formula (I), —wherein the meaning of X, W, V, Z, Y and the dotted bonds ( [symbol] ) are as given in claim 1—, characterized by
reacting a carboxylic acid of formula (II) 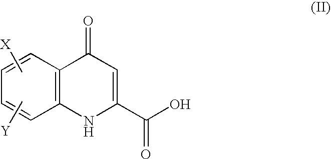
wherein the meaning of X and Y are as given in claim 1—or an active derivative thereof with an amine of formula (III) 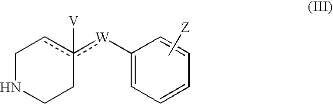
wherein the meaning of Z, V, W and the dotted bonds ( [symbol] ) are as given in claim 1—,
then transforming optionally the so obtained kynurenic acid amide derivatives of formula (I)—wherein the meaning of X, Y, W, V, Z and the dotted bonds ( [symbol] ) are as given in claim 1—into another compounds of formula (I) by introducing new substituents and/or modifying or removing the existing ones, and/or by forming salt and/or liberating the compound of formula (I) from salts, and/or by resolving the obtained racemates using optically active acids or bases by known methods.

6. Process as claimed in claim 5, characterized by reacting an active derivative of the carboxylic acid of formula (II)—wherein the meaning of X and Y are as given in claim 1—with the amine of formula (III)—wherein the meaning of Z, V, W and the dotted bonds ( [symbol] ) are as given in claim 1—preferably in the presence of a base.

7. Process as claimed in claim 5, characterized by reacting the carboxylic acid of formula (II)—wherein the meaning of X and Y are as given in claim 1—with the amine of formula (III)—wherein the meaning of Z, V, W and the dotted bonds ( [symbol] ) are as given in claim 1—in the presence of triethylamine and O-benzotriazol-1-yl-N,N,N′,N′-tetramethyl-uronium hexafluorophosphate (HBTU) in dimethylformamide.

8. Process for manufacturing pharmaceutical compositions having NR2B selective NMDA receptor antagonist effect, characterized by mixing a kynurenic acid amide derivative of formula (I)—wherein the meaning of X, W, V, Z, Y and the dotted bonds ( [symbol] ) are as given in claim 1—or optical antipodes or racemates or the pharmaceutically acceptable salts thereof as active ingredients and auxiliary materials, which are commonly used in practice, such as carriers, excipients, diluents, stabilizers, wetting or emulsifying agents, pH- and osmotic pressure-influencing, flavoring or aromatizing, as well as formulation-promoting or formulation-providing additives.

9. Method of treatment and alleviation of symptoms of the following diseases of mammals—including human—traumatic injury of brain or spinal cord, human immunodeficiency virus (IV) related neuronal injury, amyotrophic lateral sclerosis, tolerance and/or dependence to opioid treatment of pain, withdrawal syndromes of e.g. alcohol, opioids or cocaine, ischemic CNS disorders, chronic neurodegenerative disorders, such as Alzheimer's disease, Parkinson's disease, Huntington's disease, pain and chronic pain states, such as neuropathic pain or cancer related pain, epilepsy, anxiety, depression, migraine, psychosis, muscular spasm, dementia of various origin, hypoglycemia, degenerative disorders of the retina, glaucoma, asthma, tinnitus, aminoglycoside antibiotic-induced hearing loss, characterized by administering effective amount/amounts of a kynurenic acid amide derivative of formula (I)—wherein the meaning of X, W, V, Z, Y and the dotted bonds ( [symbol] )
are as given in claim 1—or optical antipodes or racemates or the pharmaceutically acceptable salts thereof as such or combined with carriers, filling materials and the like usually applied in pharmaceuticals to the mammal to be treated.

10. Use of a kynurenic acid amide derivative of formula (I)—wherein the meaning of X, W, V, Z, Y and the dotted bonds ( [symbol] ) are as given in claim 1—and/or optical antipodes or racemates and/or pharmaceutically acceptable salts thereof for the preparation of a pharmaceutical for the treatment and alleviation of symptoms of the following diseases in a mammals, including humans: traumatic injury of brain or spinal cord, human immunodeficiency virus (HIV) related neuronal injury, amyotrophic lateral sclerosis, tolerance and/or dependence to opioid treatment of pain, withdrawal syndromes of e. g. alcohol, opioids or cocaine; ischemic CNS disorders, chronic neurodegenerative disorders, such as Alzheimer's disease, Parkinson's disease, Huntington's disease, pain and chronic pain states, such as neuropathic pain or cancer related pain, epilepsy, anxiety, depression, migraine, psychosis, muscular spasm, dementia of various origin, hypoglycemia, degenerative disorders of the retina, glaucoma, asthma, tinnitus, aminoglycoside antibiotic-induced hearing loss.
